SEQUENCE LISTING (180 sequences)

SEQ ID NO: 1 - protein, 8 aa, human
HWDFAWPW

SEQ ID NO: 2 - protein, 8 aa, human
FWGLWPWE

SEQ ID NO: 3 - protein, 5 aa, Artificial Sequence
  obtained from a phage synthetic peptide library
QKRAA

SEQ ID NO: 4 - protein, 5 aa, Artificial Sequence
  obtained from a phage synthetic peptide library
RRRAA

SEQ ID NO: 5 - protein, 5 aa, vesicular stomatitis virus
KFERQ

SEQ ID NO: 6 - protein, 8 aa, vesicular stomatitis virus
RGYVYQGL

SEQ ID NO: 7 - protein, 7 aa, Artificial Sequence
  obtained from a phage synthetic peptide library
YTLVQPL

SEQ ID NO: 8 - protein, 7 aa, Artificial Sequence
  obtained from a phage synthetic peptide library
TPDITPK

SEQ ID NO: 9 - protein, 7 aa, Artificial Sequence
  obtained from a phage synthetic peptide library
TYPDLRY

SEQ ID NO: 10 - protein, 7 aa, Artificial Sequence
  obtained from a phage synthetic peptide library
DRTHATS

SEQ ID NO: 11 - protein, 7 aa, Artificial Sequence
  obtained from a phage synthetic peptide library
MSTTFYS

SEQ ID NO: 12 - protein, 7 aa, Artificial Sequence
  obtained from a phage synthetic peptide library
YQHAVQT

SEQ ID NO: 13 - protein, 7 aa, Artificial Sequence
  obtained from a phage synthetic peptide library
FPFSAST

SEQ ID NO: 14 - protein, 7 aa, Artificial Sequence
  obtained from a phage synthetic peptide library
SSFPPLD

SEQ ID NO: 15 - protein, 7 aa, Artificial Sequence
  obtained from a phage synthetic peptide library
MAPSPPH

SEQ ID NO: 16 - protein, 7 aa, Artificial Sequence
  obtained from a phage synthetic peptide library
SSFPDLL

SEQ ID NO: 17 - protein, 7 aa, Artificial Sequence
  obtained from a phage synthetic peptide library
HSYNRLP

SEQ ID NO: 18 - protein, 7 aa, Artificial Sequence
  obtained from a phage synthetic peptide library
HLTHSQR

SEQ ID NO: 19 - protein, 7 aa, Artificial Sequence
  obtained from a phage synthetic peptide library
QAAQSRS

SEQ ID NO: 20 - protein, 7 aa, Artificial Sequence
  obtained from a phage synthetic peptide library
FATHHIG

SEQ ID NO: 21 - protein, 7 aa, Artificial Sequence
  obtained from a phage synthetic peptide library
SMPEPLI

SEQ ID NO: 22 - protein, 7 aa, Artificial Sequence
  obtained from a phage synthetic peptide library
IPRYHLI

SEQ ID NO: 23 - protein, 7 aa, Artificial Sequence
  obtained from a phage synthetic peptide library
SAPHMTS

SEQ ID NO: 24 - protein, 7 aa, Artificial Sequence
  obtained from a phage synthetic peptide library
KAPVWAS

SEQ ID NO: 25 - protein, 7 aa, Artificial Sequence
  obtained from a phage synthetic peptide library
LPHWLLI

SEQ ID NO: 26 - protein, 7 aa, Artificial Sequence
  obtained from a phage synthetic peptide library
ASAGYQI

SEQ ID NO: 27 - protein, 7 aa, Artificial Sequence
  obtained from a phage synthetic peptide library
VTPKTGS

SEQ ID NO: 28 - protein, 7 aa, Artificial Sequence
  obtained from a phage synthetic peptide library
EHPMPVL

SEQ ID NO: 29 - protein, 7 aa, Artificial Sequence
  obtained from a phage synthetic peptide library
VSSFVTS

SEQ ID NO: 30 - protein, 7 aa, Artificial Sequence
  obtained from a phage synthetic peptide library
STHFTWP

SEQ ID NO: 31 - protein, 7 aa, Artificial Sequence
  obtained from a phage synthetic peptide library
GQWWSPD

SEQ ID NO: 32 - protein, 7 aa, Artificial Sequence
  obtained from a phage synthetic peptide library
GPPHQDS

SEQ ID NO: 33 - protein, 7 aa, Artificial Sequence
  obtained from a phage synthetic peptide library
NTLPSTI

SEQ ID NO: 34 - protein, 7 aa, Artificial Sequence
  obtained from a phage synthetic peptide library
HQPSRWV

SEQ ID NO: 35 - protein, 7 aa, Artificial Sequence
  obtained from a phage synthetic peptide library
YGNPLQP

SEQ ID NO: 36 - protein, 7 aa, Artificial Sequence
  obtained from a phage synthetic peptide library
FHWWWQP

SEQ ID NO: 37 - protein, 7 aa, Artificial Sequence
  obtained from a phage synthetic peptide library
ITLKYPL

SEQ ID NO: 38 - protein, 7 aa, Artificial Sequence
  obtained from a phage synthetic peptide library
FHWPWLF

SEQ ID NO: 39 - protein, 7 aa, Artificial Sequence
  obtained from a phage synthetic peptide library
TAQDSTG

SEQ ID NO: 40 - protein, 7 aa, Artificial Sequence
  obtained from a phage synthetic peptide library
FHWWWQP

SEQ ID NO: 41 - protein, 7 aa, Artificial Sequence
  obtained from a phage synthetic peptide library
FHWWDWW

SEQ ID NO: 42 - protein, 7 aa, Artificial Sequence
  obtained from a phage synthetic peptide library
EPFFRMQ

SEQ ID NO: 43 - protein, 7 aa, Artificial Sequence
  obtained from a phage synthetic peptide library
TWWLNYR

SEQ ID NO: 44 - protein, 7 aa, Artificial Sequence
  obtained from a phage synthetic peptide library
FHWWWQP

SEQ ID NO: 45 - protein, 7 aa, Artificial Sequence
  obtained from a phage synthetic peptide library
QPSHLRW

SEQ ID NO: 46 - protein, 7 aa, Artificial Sequence
  obtained from a phage synthetic peptide library
SPASPVY

SEQ ID NO: 47 - protein, 7 aa, Artificial Sequence
  obtained from a phage synthetic peptide library
FHWWWQP

SEQ ID NO: 48 - protein, 7 aa, Artificial Sequence
  obtained from a phage synthetic peptide library
HPSNQAS

SEQ ID NO: 49 - protein, 7 aa, Artificial Sequence
  obtained from a phage synthetic peptide library
NSAPRPV

SEQ ID NO: 50 - protein, 7 aa, Artificial Sequence
  obtained from a phage synthetic peptide library
QLWSIYP

SEQ ID NO: 51 - protein, 7 aa, Artificial Sequence
  obtained from a phage synthetic peptide library
SWPFFDL

SEQ ID NO: 52 - protein, 7 aa, Artificial Sequence
  obtained from a phage synthetic peptide library
DTTLPLH

SEQ ID NO: 53 - protein, 7 aa, Artificial Sequence
  obtained from a phage synthetic peptide library
WHWQMLW

SEQ ID NO: 54 - protein, 7 aa, Artificial Sequence
  obtained from a phage synthetic peptide library
DSFRTPV

SEQ ID NO: 55 - protein, 7 aa, Artificial Sequence
  obtained from a phage synthetic peptide library
TSPLSLL

SEQ ID NO: 56 - protein, 7 aa, Artificial Sequence
  obtained from a phage synthetic peptide library
AYNYVSD

SEQ ID NO: 57 - protein, 7 aa, Artificial Sequence
  obtained from a phage synthetic peptide library
RPLHDPM

SEQ ID NO: 58 - protein, 7 aa, Artificial Sequence
  obtained from a phage synthetic peptide library
WPSTTLF

SEQ ID NO: 59 - protein, 7 aa, Artificial Sequence
  obtained from a phage synthetic peptide library
ATLEPVR

SEQ ID NO: 60 - protein, 7 aa, Artificial Sequence
  obtained from a phage synthetic peptide library
SMTVLRP

SEQ ID NO: 61 - protein, 7 aa, Artificial Sequence
  obtained from a phage synthetic peptide library
QIGAPSW

SEQ ID NO: 62 - protein, 7 aa, Artificial Sequence
  obtained from a phage synthetic peptide library
APDLYVP

SEQ ID NO: 63 - protein, 7 aa, Artificial Sequence
  obtained from a phage synthetic peptide library
RMPPLLP

SEQ ID NO: 64 - protein, 7 aa, Artificial Sequence
  obtained from a phage synthetic peptide library
AKATPEH

SEQ ID NO: 65 - protein, 7 aa, Artificial Sequence
  obtained from a phage synthetic peptide library
TPPLRIN

SEQ ID NO: 66 - protein, 7 aa, Artificial Sequence
  obtained from a phage synthetic peptide library
LPIHAPH

SEQ ID NO: 67 - protein, 7 aa, Artificial Sequence
  obtained from a phage synthetic peptide library
DLNAYTH

SEQ ID NO: 68 - protein, 7 aa, Artificial Sequence
  obtained from a phage synthetic peptide library
VTLPNFH

SEQ ID NO: 69 - protein, 7 aa, Artificial Sequence
  obtained from a phage synthetic peptide library
NSRLPTL

SEQ ID NO: 70 - protein, 7 aa, Artificial Sequence
  obtained from a phage synthetic peptide library
YPHPSRS

SEQ ID NO: 71 - protein, 7 aa, Artificial Sequence
  obtained from a phage synthetic peptide library
GTAHFMY

SEQ ID NO: 72 - protein, 7 aa, Artificial Sequence
  obtained from a phage synthetic peptide library
YSLLPTR

SEQ ID NO: 73 - protein, 7 aa, Artificial Sequence
  obtained from a phage synthetic peptide library
LPRRTLL

SEQ ID NO: 74 - protein, 7 aa, Artificial Sequence
  obtained from a phage synthetic peptide library
TSTLLWK

SEQ ID NO: 75 - protein, 7 aa, Artificial Sequence
  obtained from a phage synthetic peptide library
TSDMKPH

SEQ ID NO: 76 - protein, 7 aa, Artificial Sequence
  obtained from a phage synthetic peptide library
TSSYLAL

SEQ ID NO: 77 - protein, 7 aa, Artificial Sequence
  obtained from a phage synthetic peptide library
NLYGPHD

SEQ ID NO: 78 - protein, 7 aa, Artificial Sequence
  obtained from a phage synthetic peptide library
LETYTAS

SEQ ID NO: 79 - protein, 7 aa, Artificial Sequence
  obtained from a phage synthetic peptide library
AYKSLTQ

SEQ ID NO: 80 - protein, 7 aa, Artificial Sequence
  obtained from a phage synthetic peptide library
STSVYSS

SEQ ID NO: 81 - protein, 7 aa, Artificial Sequence
  obtained from a phage synthetic peptide library
EGPLRSP

SEQ ID NO: 82 - protein, 7 aa, Artificial Sequence
  obtained from a phage synthetic peptide library
TTYHALG

SEQ ID NO: 83 - protein, 7 aa, Artificial Sequence
  obtained from a phage synthetic peptide library
VSIGHPS

SEQ ID NO: 84 - protein, 7 aa, Artificial Sequence
  obtained from a phage synthetic peptide library
THSHRPS

SEQ ID NO: 85 - protein, 7 aa, Artificial Sequence
  obtained from a phage synthetic peptide library
ITNPLTT

SEQ ID NO: 86 - protein, 7 aa, Artificial Sequence
  obtained from a phage synthetic peptide library
SIQAHHS

SEQ ID NO: 87 - protein, 7 aa, Artificial Sequence
  obtained from a phage synthetic peptide library
LNWPRVL

SEQ ID NO: 88 - protein, 7 aa, Artificial Sequence
  obtained from a phage synthetic peptide library
YYYAPPP

SEQ ID NO: 89 - protein, 7 aa, Artificial Sequence
  obtained from a phage synthetic peptide library
SLWTRLP

SEQ ID NO: 90 - protein, 7 aa, Artificial Sequence
  obtained from a phage synthetic peptide library
NVYHSSL

SEQ ID NO: 91 - protein, 7 aa, Artificial Sequence
  obtained from a phage synthetic peptide library
NSPHPPT

SEQ ID NO: 92 - protein, 7 aa, Artificial Sequence
  obtained from a phage synthetic peptide library
VPAKPRH

SEQ ID NO: 93 - protein, 7 aa, Artificial Sequence
  obtained from a phage synthetic peptide library
HNLHPNR

SEQ ID NO: 94 - protein, 7 aa, Artificial Sequence
  obtained from a phage synthetic peptide library
YTTHRWL

SEQ ID NO: 95 - protein, 7 aa, Artificial Sequence
  obtained from a phage synthetic peptide library
AVTAAIV

SEQ ID NO: 96 - protein, 7 aa, Artificial Sequence
  obtained from a phage synthetic peptide library
TLMHDRV

SEQ ID NO: 97 - protein, 7 aa, Artificial Sequence
  obtained from a phage synthetic peptide library
TPLKVPY

SEQ ID NO: 98 - protein, 7 aa, Artificial Sequence
  obtained from a phage synthetic peptide library
FTNQQYH

SEQ ID NO: 99 - protein, 7 aa, Artificial Sequence
  obtained from a phage synthetic peptide library
SHVPSMA

SEQ ID NO: 100 - protein, 7 aa, Artificial Sequence
  obtained from a phage synthetic peptide library
HTTVYGA

SEQ ID NO: 101 - protein, 7 aa, Artificial Sequence
  obtained from a phage synthetic peptide library
TETPYPT

SEQ ID NO: 102 - protein, 7 aa, Artificial Sequence
  obtained from a phage synthetic peptide library
LTTPFSS

SEQ ID NO: 103 - protein, 7 aa, Artificial Sequence
  obtained from a phage synthetic peptide library
GVPLTMD

SEQ ID NO: 104 - protein, 7 aa, Artificial Sequence
  obtained from a phage synthetic peptide library
KLPTVLR

SEQ ID NO: 105 - protein, 7 aa, Artificial Sequence
  obtained from a phage synthetic peptide library
CRFHGNR

SEQ ID NO: 106 - protein, 7 aa, Artificial Sequence
  obtained from a phage synthetic peptide library
YTRDFEA

SEQ ID NO: 107 - protein, 7 aa, Artificial Sequence
  obtained from a phage synthetic peptide library
SSAAGPR

SEQ ID NO: 108 - protein, 7 aa, Artificial Sequence
  obtained from a phage synthetic peptide library
SLIQYSR

SEQ ID NO: 109 - protein, 7 aa, Artificial Sequence
  obtained from a phage synthetic peptide library
DALMWPX

SEQ ID NO: 110 - protein, 7 aa, Artificial Sequence
  obtained from a phage synthetic peptide library
SSXSLYI

SEQ ID NO: 111 - protein, 7 aa, Artificial Sequence
  obtained from a phage synthetic peptide library
FNTSTRT

SEQ ID NO: 112 - protein, 7 aa, Artificial Sequence
  obtained from a phage synthetic peptide library
TVQHVAF

SEQ ID NO: 113 - protein, 7 aa, Artificial Sequence
  obtained from a phage synthetic peptide library
DYSFPPL

SEQ ID NO: 114 - protein, 7 aa, Artificial Sequence
  obtained from a phage synthetic peptide library
VGSMESL

SEQ ID NO: 115 - protein, 7 aa, Artificial Sequence
  obtained from a phage synthetic peptide library
FXPMIXS

SEQ ID NO: 116 - protein, 7 aa, Artificial Sequence
  obtained from a phage synthetic peptide library
APPRVTM

SEQ ID NO: 117 - protein, 7 aa, Artificial Sequence
  obtained from a phage synthetic peptide library
IATKTPK

SEQ ID NO: 118 - protein, 7 aa, Artificial Sequence
  obtained from a phage synthetic peptide library
KPPLFQI

SEQ ID NO: 119 - protein, 7 aa, Artificial Sequence
  obtained from a phage synthetic peptide library
YHTAHNM

SEQ ID NO: 120 - protein, 7 aa, Artificial Sequence
  obtained from a phage synthetic peptide library
SYIQATH

SEQ ID NO: 121 - protein, 7 aa, Artificial Sequence
  obtained from a phage synthetic peptide library
SSFATFL

SEQ ID NO: 122 - protein, 7 aa, Artificial Sequence
  obtained from a phage synthetic peptide library
TTPPNFA

SEQ ID NO: 123 - protein, 7 aa, Artificial Sequence
  obtained from a phage synthetic peptide library
ISLDPRM

SEQ ID NO: 124 - protein, 7 aa, Artificial Sequence
  obtained from a phage synthetic peptide library
SLPLFGA

SEQ ID NO: 125 - protein, 7 aa, Artificial Sequence
  obtained from a phage synthetic peptide library
NLLKTTL

SEQ ID NO: 126 - protein, 7 aa, Artificial Sequence
  obtained from a phage synthetic peptide library
DQNLPRR

SEQ ID NO: 127 - protein, 7 aa, Artificial Sequence
  obtained from a phage synthetic peptide library
SHFEQLL

SEQ ID NO: 128 - protein, 7 aa, Artificial Sequence
  obtained from a phage synthetic peptide library
TPQLHHG

SEQ ID NO: 129 - protein, 7 aa, Artificial Sequence
  obtained from a phage synthetic peptide library
APLDRIT

SEQ ID NO: 130 - protein, 7 aa, Artificial Sequence
  obtained from a phage synthetic peptide library
FAPLIAH

SEQ ID NO: 131 - protein, 7 aa, Artificial Sequence
  obtained from a phage synthetic peptide library
SWIQTFM

SEQ ID NO: 132 - protein, 7 aa, Artificial Sequence
  obtained from a phage synthetic peptide library
NTWPHMY

SEQ ID NO: 133 - protein, 7 aa, Artificial Sequence
  obtained from a phage synthetic peptide library
EPLPTTL

SEQ ID NO: 134 - protein, 7 aa, Artificial Sequence
  obtained from a phage synthetic peptide library
HGPHLFN

SEQ ID NO: 135 - protein, 7 aa, Artificial Sequence
  obtained from a phage synthetic peptide library
YLNSTLA

SEQ ID NO: 136 - protein, 7 aa, Artificial Sequence
  obtained from a phage synthetic peptide library
HLHSPSG

SEQ ID NO: 137 - protein, 7 aa, Artificial Sequence
  obtained from a phage synthetic peptide library
TLPHRLN

SEQ ID NO: 138 - protein, 7 aa, Artificial Sequence
  obtained from a phage synthetic peptide library
SSPREVH

SEQ ID NO: 139 - protein, 7 aa, Artificial Sequence
  obtained from a phage synthetic peptide library
NQVDTAR

SEQ ID NO: 140 - protein, 7 aa, Artificial Sequence
  obtained from a phage synthetic peptide library
YPTPLLT

SEQ ID NO: 141 - protein, 7 aa, Artificial Sequence
  obtained from a phage synthetic peptide library
HPAAFPW

SEQ ID NO: 142 - protein, 7 aa, Artificial Sequence
  obtained from a phage synthetic peptide library
LLPHSSA

SEQ ID NO: 143 - protein, 7 aa, Artificial Sequence
  obtained from a phage synthetic peptide library
LETYTAS

SEQ ID NO: 144 - protein, 7 aa, Artificial Sequence
  obtained from a phage synthetic peptide library
KYVPLPP

SEQ ID NO: 145 - protein, 7 aa, Artificial Sequence
  obtained from a phage synthetic peptide library
APLALHA

SEQ ID NO: 146 - protein, 7 aa, Artificial Sequence
  obtained from a phage synthetic peptide library
YESLLTK

SEQ ID NO: 147 - protein, 7 aa, Artificial Sequence
  obtained from a phage synthetic peptide library
SHAASGT

SEQ ID NO: 148 - protein, 7 aa, Artificial Sequence
  obtained from a phage synthetic peptide library
GLATVKS

SEQ ID NO: 149 - protein, 7 aa, Artificial Sequence
  obtained from a phage synthetic peptide library
GATSFGL

SEQ ID NO: 150 - protein, 7 aa, Artificial Sequence
  obtained from a phage synthetic peptide library
KPPGPVS

SEQ ID NO: 151 - protein, 7 aa, Artificial Sequence
  obtained from a phage synthetic peptide library
TLYVSGN

SEQ ID NO: 152 - protein, 7 aa, Artificial Sequence
  obtained from a phage synthetic peptide library
HAPFKSQ

SEQ ID NO: 153 - protein, 7 aa, Artificial Sequence
  obtained from a phage synthetic peptide library
VAFTRLP

SEQ ID NO: 154 - protein, 7 aa, Artificial Sequence
  obtained from a phage synthetic peptide library
LPTRTPA

SEQ ID NO: 155 - protein, 7 aa, Artificial Sequence
  obtained from a phage synthetic peptide library
ASFDLLI

SEQ ID NO: 156 - protein, 7 aa, Artificial Sequence
  obtained from a phage synthetic peptide library
RMNTEPP

SEQ ID NO: 157 - protein, 7 aa, Artificial Sequence
  obtained from a phage synthetic peptide library
KMTPLTT

SEQ ID NO: 158 - protein, 7 aa, Artificial Sequence
  obtained from a phage synthetic peptide library
ANATPLL

SEQ ID NO: 159 - protein, 7 aa, Artificial Sequence
  obtained from a phage synthetic peptide library
TIWPPPV

SEQ ID NO: 160 - protein, 7 aa, Artificial Sequence
  obtained from a phage synthetic peptide library
QTKVMTT

SEQ ID NO: 161 - protein, 7 aa, Artificial Sequence
  obtained from a phage synthetic peptide library
NHAVFAS

SEQ ID NO: 162 - protein, 7 aa, Artificial Sequence
  obtained from a phage synthetic peptide library
LHAAXTS

SEQ ID NO: 163 - protein, 7 aa, Artificial Sequence
  obtained from a phage synthetic peptide library
TWQPYFH

SEQ ID NO: 164 - protein, 7 aa, Artificial Sequence
  obtained from a phage synthetic peptide library
APLALHA

SEQ ID NO: 165 - protein, 7 aa, Artificial Sequence
  obtained from a phage synthetic peptide library
TAHDLTV

SEQ ID NO: 166 - protein, 7 aa, Artificial Sequence
  obtained from a phage synthetic peptide library
NMTNMLT

SEQ ID NO: 167 - protein, 7 aa, Artificial Sequence
  obtained from a phage synthetic peptide library
GSGLSQD

SEQ ID NO: 168 - protein, 7 aa, Artificial Sequence
  obtained from a phage synthetic peptide library
TPIKTIY

SEQ ID NO: 169 - protein, 7 aa, Artificial Sequence
  obtained from a phage synthetic peptide library
SHLYRSS

SEQ ID NO: 170 - protein, 7 aa, Artificial Sequence
  obtained from a phage synthetic peptide library
HGQAWQF

SEQ ID NO: 171 - protein, 12 aa, Artificial Sequence
  obtained from a phage synthetic peptide library
CGSGHWDFAWPW

SEQ ID NO: 172 - protein, 12 aa, Artificial Sequence
  obtained from a phage synthetic peptide library
HWDFAWPWGSGC

SEQ ID NO: 173 - protein, 12 aa, Artificial Sequence
  obtained from a phage synthetic peptide library
CGSGFWGLWPWE

SEQ ID NO: 174 - protein, 12 aa, Artificial Sequence
  obtained from a phage synthetic peptide library
FWGLWPWEGSGC

SEQ ID NO: 175 - protein, 9 aa, Artificial Sequence
  obtained from a phage synthetic peptide library
CHWDFAWPW

SEQ ID NO: 176 - protein, 9 aa, Artificial Sequence
  obtained from a phage synthetic peptide library
HWDFAWPWC

SEQ ID NO: 177 - protein, 9 aa, Artificial Sequence
  obtained from a phage synthetic peptide library
CFWGLWPWE

SEQ ID NO: 178 - protein, 9 aa, Artificial Sequence
  obtained from a phage synthetic peptide library
FWGLWPWEC

SEQ ID NO: 179 - protein, 4 aa, Artificial Sequence
  obtained from a phage synthetic peptide library
CGSG

SEQ ID NO: 180 - protein, 22 aa, Camelus dromedarius
PQPQPKPQPQPQPKPQPKPEPE

(19) United States
(12) Patent Application Publication
(10) Pub. No.: US 2004/0052812 A1
(43) Pub. Date: Mar. 18, 2004

(54) Heat shock protein-based antiviral vaccines

(76) Inventors: Mee Hoe, Irvington, NY (US); Frank Landsberger, New York, NY (US)

Correspondence Address: KENYON & KENYON, ONE BROADWAY, NEW YORK, NY, 10004, US

(21) Appl. No.: 10/258,146

(22) PCT Filed: Apr. 17, 2001
(86) PCT No.: PCT/US01/12568

(30) Foreign Application Priority Data
Apr. 17, 2000 (US) 60197462

(51) Int. Cl. A61K 39/12

(52) U.S. Cl. 424/186.1

(57) ABSTRACT

The present invention relates to the use of non-pathogenic multi-cmponent viral particles in vaccines whcih utilize heat shock proteins to enhance the anti-viral immune response. The multi-component viral particles are covalently conjugated to one or more species of “javelin”, where javelins are molecules which form non-covalent associatiosn with heat shock proteins. In view of the role of heat shock proteins in the recognition, by the immune system, of antigens, the addition of a javelin “tether” to a multi-component viral particle facilitates complex formation between the particle and a heat shock protein and hence promotes development of an immune reaction to the particle, without requiring the identification of specific epitopes. In addition, the present invention provides for methods of preventing or ameliorating viral infections comprising administering a “javelinized” multi-component viral particle vaccine to a subject at risk of contracting a viral infection or who has already been infected. Because of the diversity of epitopes in the multi-component viral particles, a single vaccine formulation may be used to promote immunity toward multiple viral strains in subjects having various histocompatibility profiles.

### 1. INTRODUCTION

The present invention relates to vaccines to prevent or ameliorate viral infections in which a non-pathogenic multi-component viral particle is attached to a molecule, referred to as a “javelin”, which acts as a non-covalent tether to a heat shock protein.

### 2. BACKGROUND OF THE INVENTION

Because modern medicine has had, to date, limited success in treating viral infections, a more successful approach is to prevent infection from occurring in the first place. To this end, various vaccines have been developed, using different strategies to pre-empt infection and/or prevent disease progression.

Examples of categories of vaccine include live virus vaccines, where the virus has been weakened, or attenuated, such that it cannot cause disease; killed-virus vaccines; vaccines which contain one or more viral proteins; chimeric viruses whereby a non-pathogenic virus is engineered to contain genetic information encoding immunogenic peptide(s) from a disease-causing virus; and naked DNA encoding such peptides. Of the last two categories of vaccine, the non-pathogenic virus can “deliver” the immunogenic peptides by infecting host cells, and the naked DNA can be injected, for example intramuscularly, into host cells where it can be taken up and ultimately expressed as antigenic protein. The requirements for a vaccine to be effective vary from virus to virus, and depend upon, among other things, whether, and to what degree, humoral and/or cellular immunity is necessary to prevent infection, the genetic variability in the immunogenic regions of a virus, and virulence. Yet another category of vaccines uses self-replicating and self-limiting RNA (“RNA replicons”), which cause lysis of transfected cells and do not-raise the concerns associated with naked DNA vaccines, which can integrate into host chromosomes (Cheng et al., 2001, J. Virol. 75(5):2368-2376).

For some viruses vaccine development has been more successful than for others, as will be apparent from the illustrative examples to follow.

An example of a successful antiviral campaign through vaccines is the remarkable decrease in the incidence of poliomyelitis (“polio”), which had, in the first half of the last century, devastated hundreds of thousands of individuals with crippling neurological disease. The incidence of disease was abruptly curtailed by vaccination with inactivated polio virus vaccine (“IPV”) after 1955 and by live attenuated vaccine (which could be administered orally, hence “OPV”) after 1960 (Fields Virology, 1996, Third Edition, Fields et al., eds., Lippincott-Raven Press, New York, p. 694). The decrease in the number of cases was remarkable. In 1955, in the Soviet Union, 23 European countries, the United States, Canada, Australia and New Zealand, there were 76,000 reported cases of poliomyelitis; by 1967, the number of cases had fallen to only 1,013, a reduction of almost 99 percent (Id.).

The hepatitis B vaccine, available since 1982, has also been extremely effective in preventing disease. It contains recombinant protein containing a portion of hepatitis B surface antigen, and has been successful in protecting persons against acute hepatitis B infection as well as its more chronic consequences, including cirrhosis and cancer of the liver (www.cdc.gov/ncidod/diseases/hepatitis/b/faqbvax.htm, citing CDC, 1991, MMWR 40(RR-13):1-17 and Hadler et al., 1992, in Current Clinical Topics in Infectious Diseases, Remington and Swartz, eds., Blackwell Scientific Publications, pp. 282-308).

More problematic is the prevention of influenza, caused, in the human population, by Type A and B influenza viruses which mutate at such a rapid rate that the vaccine needs to be modified from year to year. Typically, the variations are modest, so that persons can carry some level of immunity from one year to the next. From time to time, however, a sufficiently different form of virus appears (that is to say, there is an “antigenic shift”) to which no one has been exposed, resulting in local epidemics or, in the worst case, global pandemics (WHO Fact Sheet No. 188, January 1998, www.who.int/inf-fs/en/fact188.html). In the last century, three pandemics occurred, in 1918, 1957, and 1968.

Within the last five years, the World Health Organization created a Task Force of Experts on Influenza who maintain surveillance of influenza infections throughout the world. Based on their observation of antigenic trends, the Task Force facilitates the development and distribution of annual vaccines. This year, the availability of the-influenza vaccine was delayed due to lower than expected production yields of the new influenza A H3N2 strain (where the H/N nomenclature refers to the hemagglutinin and neuraminidase viral proteins; www.fda.gov/cber/flu/flu2000.htm).

A new approach, focusing on a minor coat protein, M2, which is less variable than the hemagglutinin protein, has promise (Kilbourne et al., 1999, Nature Medicine 5:1119-1120; Nierynck et al., 1999, Nature Medicine 5: 1157-1163); whether or not M2-directed vaccines will be adequately clinically effective remains to be seen. In May, 1997, a new deadly form of-influenza virus (A H5N1), previously known only to infect birds, was identified in a human patient in Hong Kong. Incidence of infection with this viral type is being carefully monitored.

Mumps, which can have serious clinical consequences (particularly in adults),is another viral infection which has been difficult to prevent. A Swedish group (Nojd et al., 2001, Vaccine 19(13-14):1727-1731) reports that a previously healthy 22-year old woman suffering from chronic disease caused by the mumps virus had a pre-infection mumps antibody titer which would seem to have been sufficient to prevent infection. However, the chronic infection in this patient was caused by a different genotype of mumps virus. The authors suggest that the inability of antibodies to protect against re-infection with a heterologous mumps genotype might explain vaccine failures.

Perhaps most problematic have been attempts to develop a vaccine which protects against human immunodeficiency virus (“HIV”) infection and the development of acquired immunodeficiency syndrome (“AIDS”). An article published last year (Klein and Ho, 2000, Clin. Ther. 23:295-314) reviewed the status of the development of an HIV vaccine and concluded that at that time only two vaccine candidates were in phase m clinical trials, and data suggested that they produced an antibody response only. Several studies are cited which suggest that these approaches will be ineffective in providing any real protection from viral infection because they fail to produce a strong cellular immune response.

According to Esparza et al., 1995, Drugs 50(5):792-804, “[a] major concern for the development of broadly effective vaccines has been the extensive genetic variability which is characteristic of HIV.” A recent study (Mooij et al., 2000, J. Virol. 74(9):4017-4027) indicates that, at least as regards subunit vaccines based on the CCR5 binding envelope of HIV-1, protection achieved toward certain strains of HIV may disappear toward virulent variants.

Peters, 2000, Antivir. Chem. Chemother. 11(5):311-320 concludes that the most developed agent to date is Remune, which is a gp120 depleted whole killed HIV-1 vaccine that has been observed to induce responses (albeit smaller than desired) in CD4 count and viral load. Peters expresses optimism toward newer approaches, including recombinant canarypox vaccines like ALVAC 1452 and highly attenuated vaccinia virus vaccines, used in combination with HIV genes and peptides. According to Engelmayer et al., 2001, J. Virol. 75(5):2142-2153, whereas recombinant canarypox vectors containing HIV-1 sequences are promising vaccine candidates because they replicate poorly in human cells, they exhibit the shortcoming of inducing inconsistent and sometimes transient antigen-specific cytotoxic T cell responses. Engelmayer et al. suggest, as a potential solution to this problem, targeting canarypox virus vectors to professional antigen-presenting cells, such as dendritic cells. In summary, to date, the prior art has not developed a safe and effective vaccine toward HIV infection.

Thus there remains a need for the development of vaccines which may be efficiently prepared and which may be used to induce both humoral and cellular immunity.

### 3. SUMMARY OF THE INVENTION

The present invention relates to the use of non-pathogenic multi-component viral particles in vaccines which utilize heat shock proteins to enhance the anti-viral immune response. The multi-component viral particles are covalently conjugated to one or more species of “javelin”, where javelins are molecules which form non-covalent associations with heat shock proteins. In view of the role of heat shock proteins in the recognition, by the immune system, of antigens, the addition of a javelin “tether” to a multi-component viral particle facilitates complex formation between the particle and a heat shock protein and hence promotes development of an immune reaction to the particle, without requiring the identification of specific epitopes.

In addition, the present invention provides for methods of preventing or ameliorating viral infections comprising administering a “javelinized” multi-component viral particle vaccine to a subject at risk of contracting a viral infection or who has already been infected. Because of the diversity of epitopes in the multi-component viral particles, a single vaccine formulation may be used to promote immunity toward multiple viral strains in subjects having various histocompatibility profiles.

### 4. DESCRIPTION OF THE FIGURES

FIG. 1. Illustration of HIV particle showing relationship between surface and interior proteins and host-derived lipid bilayer coat (source: www.med.sc.edu:85/lecture/hivstruct.gif).

FIG. 2. Diagram of mature HIV particle (source: www.med.sc.edu:85/lecture/HIVmod2.GIF).

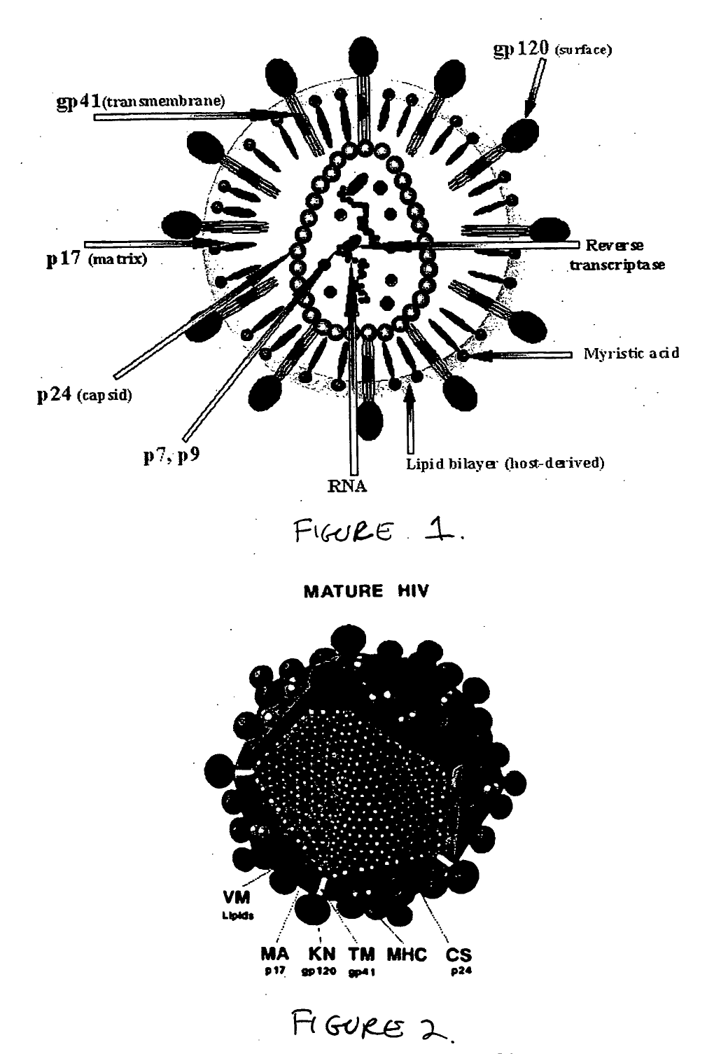

### 5. DETAILED DESCRIPTION OF THE INVENTION

The present invention relates to vaccines and to methods for inducing, in a subject, immunity to a virus, whereby non-pathogenic multi-component viral particles are covalently conjugated to one or more species of “javelin” molecules which non-covalently bind the multi-component viral particles to heat shock proteins. Accordingly, this section describes various viral components and javelins, how they may be linked, and how the ability of such complexes to associate with heat shock protein and facilitate an immune response may be tested. For purposes of clarity of presentation, and not by way of limitation, this section of the specification is divided into the following subsections:

1) viral components;

2) “javelin” tethers;

3) methods of linking viral components to javelins;

4) assays to determine lack of pathogenicity;

5) assays to determine immunogenicity;

6) compositions of the invention; and

7) methods of inducing immunity.

### 5.1 VIRAL COMPONENTS

The present invention relates to a multi-component viral particle covalently linked to a javelin tether, defined in the following subsection, such that the resulting structure is able to non-covalently bind to one or more heat shock proteins.

The multi-component viral particle is defined herein as a plurality of viral components which are physically joined, so as to be distinguishable from an isolated viral protein. The viral components within the particle may be joined by covalent, non-covalent, ionic, or Van der Waals bonds or forces, or a combination of any of these bonds or forces. Assembly of the particle may occur in nature or may be effected by genetic engineering or chemical techniques.

In some embodiments of the invention, the viral components may originate from the same strain or a particular type of virus; for example, all the viral proteins may originate from the same strain of HIV, influenza virus, or human papilloma virus. In other embodiments, the viral components may originate from different strains of the same type of virus, for example, from different strains of HIV, or from different strains of human papilloma virus (e.g., strains 16, 18, and/or 33). In further embodiments of the invention, the viral components may originate from different types of virus, for example, for use in humans, polio virus and respiratory syncytial virus, or, for use in dogs, rabies and canine distemper virus.

As a non-limiting example, because HIV infection may be facilitated by infection with Herpes Simplex Virus (“HSV”; Mosca et al., 1988, Nature 331:122; Mosca et al., 1987, Proc. Natl. Acad. Sci. U.S.A. 84:7408-7412; Mosca et al., 1987, Nature 325:67-70), an HIV viral component (e.g.. a subunit protein such as gp160 or gp120) may be combined with a HSV viral component (e.g., an inactivated or attenuated strain of HSV). Because of the association between Kaposi's sarcoma nd HSV 8, in preferred non-limiting embodiments of the invention the HSV component is derived from HSV 8. Similarly, an HIV viral component may be combined with other viral or non-viral components (e.g. a mycobacterial component). In this regard, it should be noted that multi-component viral particles may comprise components derived from non-viral sources, such as components from bacteria, yeast, protozoa, or eukaryotic cells or non-naturally occurring synthetic components.

The viral components may include members from the same biochemical category of molecules; for example, the viral components may all be viral proteins. In such embodiments, the viral proteins may be full-length viral proteins, portions thereof, or fusion proteins comprising viral proteins. Non-limiting examples of such combinations of proteins include: for use in influenza vaccines, multi-component particles comprising a plurality of serotypes of hemagglutinin proteins, or portions thereof, or comprising a single serotype of hemagglutinin and an M2 protein, or portions thereof, or comprising a plurality of serotypes of hemagglutinin and an M2 protein, or portions thereof, or any of the foregoing further comprising neuraminidase protein, or portions thereof; for use in HIV vaccines, multi-component particles comprising gp120 and/or gp160, or portions thereof, or comprising env and gag gene products, or portions thereof, or comprising env and protease gene products, or portions thereof, or comprising env, gag and protease gene products, or portions thereof, including but not limited to matrix protein (p17), capsid protein, nucleocapsid protein (p24), p6, p7, protease protein, reverse transcriptase, integrase, gp41, Vif, Vpr, nef, tat, rev, and Vpe, or portions thereof; and, for use in human papilloma virus vaccines, multi-component particles comprising the L1 major capsid protein, or a portion thereof, and/or the L2 protein, or portions thereof.

Alternatively, the viral components may include members from different biochemical categories of molecule; for example, protein and lipid, or protein and nucleic acid, or protein, lipid, and nucleic acid. The lipid may, for instance, be derived from the membrane of an infected cell, as in the case of an enveloped virus. In this regard, the multi-component viral particle may be, for example, a form of virus lacking one or more component critical to infection and/or replication, such as a viral capsid lacking the viral genome, or an otherwise intact viral particle lacking a protein necessary for viral replication or for host cell entry. One non-limiting example of a class of such particles are virus-like particles (“VLPs”) currently being developed as human papillomavirus vaccines (Schiller and Lowy, 2000, J. Natl. Cancer Inst. Monogr. 2000 (28):50-54).

For example, a multi-component viral particle may be a “mutant” form of virus which has been either engineered or isolated from nature and which lacks one or more viral protein which operates, in the native virus, to assist the virus in evading the host immune system. The omitted viral protein(s) may naturally fumction to inhibit or modulate the host humoral and/or cellular immune response or to interfere with chemokine production or function, or with apoptotic cell death (Alcami et al., 2000, Immunol. Today 21:447-455) The multi-component viral particle lacking such protein(s) may be more successful at inducing a protective immune response.

In particular, non-limiting embodiments, the multi-component viral particle of the invention is an attenuated virus. An “attenuated virus” is a virus derived from a pathogenic parent strain but which has been treated in a manner which renders the virus capable of infection but non-pathogenic, or only capable of infection under certain conditions. Attenuation may be achieved by a number of techniques known in the art, such as by chemical treatment, by passaging under particular conditions, or by genetic engineering. See, for example, U.S. Pat. No. 3,981,772 by Poverenny et al., which relates to attenuation using an aminomethylol compound, or U.S. Pat. No. 6,077,514 by Maasab et al., which relates to specific cold-adapted or temperature sensitive strains of respiratory syncytial virus (“RSV”).

In other, non-limiting embodiments of the invention, the multi-component viral particle of the invention is a killed virus. A “killed virus” is a pathogenic virus which has been treated in a manner which renders it incapable of infection. Inactivation methods known in the art utilize physical and/or chemical agents such as heat and/or formalin, β-propiolactone and ethylenimines, as well as other amines and amides (Miyamae, 1994, Microbiol. Immunol. 38:937-941). See, for example, U.S. Pat. No. 5,106,619 by Wiesehahn et al., which relates to psolaren-mediated inactivation in a non-oxidizing atmosphere, which is applicable to enveloped, as well as non-enveloped, viruses.

The multi-component viral particles of the invention are nonpathogenic. The term “non-pathogenic” as defined herein means that a multi-component viral particle does not have a pathogenic effect when exposed to a cell or organism which is typically susceptible to a pathogenic effect caused by the virus or viruses from which the multi-component viral particle is derived.

A multi-component viral particle is “derived” from a particular virus if it is either prepared from a native virus particle or produced chemically or using genetic engineering techniques (e.g., by expressing a viral protein encoded by a cloned nucleic acid of the virus) so as to duplicate, or to produce in a modified form, a constituent of the native virus.

A “pathogenic effect” would be recognized by the skilled artisan, and would include, for example, histologic changes such as a cytopathic effect or cell fusion in cell culture as well as clinical symptoms, which may be moderate to severe, in an organism. Mild symptoms, such as “flu-like” symptoms often associated with the administration of attenuated viral vaccines currently in use, would still fall within the definition of “non-pathogenic” used herein. Nor does the term “non-pathogenic” preclude infection or virus replication.

Accordingly, the multi-component viral particles of the invention may be derived from viruses including, but not limited to, the following: viruses having a genome comprised of double stranded DNA, such as adenoviruses, herpes viruses such as herpes simplex viruses (I and II) and feline herpes viruses, papovaviruses such as polyoma virus and papilloma virus, poxviruses such as smallpox virus and vaccinia virus and hepadnaviridae (which have a genome which is partially single stranded) such as hepatitis B; viruses having a genome consisting essentially of single stranded DNA, such as parvoviruses (e.g., canine parvovirus); viruses having a genome comprised of single stranded RNA, such as calciviruses, coronaviruses, myxoviruses such as influenza virus, paramyxoviruses such as measles virus, mumps virus, Newcastle disease virus, respiratory syncytial virus, and canine distemper virus, picomaviruses such as polio virus, retroviruses such as HIV and feline leukemia virus, rhabdoviruses such as vesicular stomatitis virus and rabies virus, and flaviviruses such as hepatitis C virus, the virus which causes yellow fever, and the viruses associated with tick-bome encephalitis; and viruses having a genome comprised of double stranded RNA such as orbiviruses and reoviruses.

### 5.2 JAVELIN TETHERS

The term “javelin” as used herein refers to a molecule which, when covalently linked to a multi-component viral particle, acts as a tether, creating a non-covalent physical association between the multi-component viral particle and the heat shock protein.

The javelin (alternatively referred to herein as a “javelin molecule”) may be a member of any class of biochemical molecule or combination thereof, but is preferably a peptide (“javelin peptide”) or a peptidomimetic compound. The structures ofjavelins will vary, at least to some degree, depending on the particular heat shock protein to which each javelin binds. It should be noted, however, that because a number of heat shock proteins act as molecular chaperones in the process of protein folding, they are typically capable of binding to a variety ofjavelin molecules. Suitable javelin molecules, and methods for identifying further javelin molecules, are described in co-pending International Patent Application No. PCT/US98/22335 by Sloan-Kettering Institute for Cancer Research, Rothman et al., inventors, International Publication No. WO99/22761, incorporated by reference in its entirety herein.

Accordingly, the javelin to be covalently linked to a multi-component viral particle is chosen based on the particular heat shock protein or heat shock proteins to which it is intended to bind. Such heat shock protein may be any known or yet to be identified heat shock protein or portion thereof, or any fusion protein comprising at least a portion of a heat shock protein. The term “heat shock protein”, as used herein, refers to stress proteins (including homologs thereof expressed constitutively), including, but not limited to, gp96, gp170, hsp90, BiP, hsp70, hsp60, hsp40, hsc70, and hsp10 and families of proteins with homologies to stress proteins.

In particular, non-limiting embodiments of the invention, javelins may have amino acid compositions which comprise a substantial proportion of hydrophobic amino acids such as phenylalanine and tryptophan, and to a lesser extent, leucine and/or a substantial number of serine, threonine, or proline residues. In particular, nonlimiting embodiments, javelins of the invention may comprise amino acid sequences which have the general description hydrophobic-basic-hydrophobic-hydrophobic-hydrophobic; Ser/Thr-hydrophobic-hydrophobic-Ser/Thr; Ser/Thr-Ser/Thr-hydrophobic-hydrophobic-Ser/Thr-Ser/Thr; and Ser/Thr-Ser/Thr hydrophobic-hydrophobic-hydrophobic. Alternatively, javelins may comprise heat shock binding peptides as described in Blond-Elguindi et al., 1993, Cell 75:717-728, including the consensus sequence hydrophobic-(Trp/X)-hydrophobic-X-hydrophobic-X-hydrophobic and the specific peptides His Trp Asp Phe Ala Trp Pro Trp (SEQ ID NO: 1) and Phe Trp Gly Leu Trp Pro Trp Glu (SEQ ID NO: 2); Auger et al., 1996, Nature Med. 2:306-310, including Gln Lys Arg Ala Ala (SEQ ID NO: 3) and Arg Arg Arg Ala Ala (SEQ ID NO: 4); Flynn et al., 1989, Science 245:385-390; Gragerov et al., 1994, J. Mol. Biol. 235:848-854; Terlecky et al., 1992, J. Biol. Chem. 267:9202-9202, Lys Phe Glu Arg Gln (SEQ ID NO: 5); and Nieland et al., 1996, Proc. Natl. Acad. Sci. U.S.A. 93:6135-6139, including the VSV8 peptide, Arg Gly Tyr Val Tyr Gln Gly Leu (SEQ ID NO: 6). In preferred non-limiting embodiments, javelins of the invention may have a length of 4-50 amino acid residues, and more preferably 7-20 amino acid residues.

In specific, non-limiting embodiments, the following amino acid sequences, discussed more fully in International Patent Application No. PCT/US98/22335, cited supra, may be covalently linked to multi-component viral particles according to the invention:

| | |
|---|---|
| Tyr Thr Leu Val Gln Pro Leu; | (SEQ ID NO:7) |
| Thr Pro Asp Ile Thr Pro Lys; | (SEQ ID NO:8) |
| Thr Tyr Pro Asp Leu Arg Tyr; | (SEQ ID NO:9) |
| Asp Arg Thr His Ala Thr Ser; | (SEQ ID NO:10) |
| Met Ser Thr Thr Phe Tyr Ser; | (SEQ ID NO:11) |
| Tyr Gln His Ala Val Gln Thr; | (SEQ ID NO:12) |
| Phe Pro Phe Ser Ala Ser Thr; | (SEQ ID NO:13) |
| Ser Ser Phe Pro Pro Leu Asp; | (SEQ ID NO:14) |
| Met Ala Pro Ser Pro Pro His; | (SEQ ID NO:15) |
| Ser Ser Phe Pro Asp Leu Leu; | (SEQ ID NO:16) |
| His Ser Tyr Asn Arg Leu Pro; | (SEQ ID NO:17) |
| His Leu Thr His Ser Gln Arg; | (SEQ ID NO:18) |
| Gln Ala Ala Gln Ser Arg Ser; | (SEQ ID NO:19) |
| Phe Ala Thr His His Ile Gly; | (SEQ ID NO:20) |
| Ser Met Pro Glu Pro Leu Ile; | (SEQ ID NO:21) |
| Ile Pro Arg Tyr His Leu Ile; | (SEQ ID NO:22) |
| Ser Ala Pro His Met Thr Ser; | (SEQ ID NO:23) |
| Lys Ala Pro Val Trp Ala Ser; | (SEQ ID NO:24) |
| Leu Pro His Trp Leu Leu Ile; | (SEQ ID NO:25) |
| Ala Ser Ala Gly Tyr Gln Ile; | (SEQ ID NO:26) |
| Val Thr Pro Lys Thr Gly Ser; | (SEQ ID NO:27) |
| Glu His Pro Met Pro Val Leu; | (SEQ ID NO:28) |
| Val Ser Ser Phe Val Thr Ser; | (SEQ ID NO:29) |
| Ser Thr His Phe Thr Trp Pro; | (SEQ ID NO:30) |
| Gly Gln Trp Trp Ser Pro Asp; | (SEQ ID NO:31) |
| Gly Pro Pro His Gln Asp Ser; | (SEQ ID NO:32) |
| Asn Thr Leu Pro Ser Thr Ile; | (SEQ ID NO:33) |
| His Gln Pro Ser Arg Trp Val; | (SEQ ID NO:34) |
| Tyr Gly Asn Pro Leu Gln Pro; | (SEQ ID NO:35) |
| Phe His Trp Trp Trp Gln Pro; | (SEQ ID NO:36) |
| Ile Thr Leu Lys Tyr Pro Leu; | (SEQ ID NO:37) |
| Phe His Trp Pro Trp Leu Phe; | (SEQ ID NO:38) |
| Thr Ala Gln Asp Ser Thr Gly; | (SEQ ID NO:39) |
| Phe His Trp Trp Trp Gln Pro; | (SEQ ID NO:40) |
| Phe His Trp Trp Asp Trp Trp; | (SEQ ID NO:41) |
| Glu Pro Phe Phe Arg Met Gln; | (SEQ ID NO:42) |
| Thr Trp Trp Leu Asn Tyr Arg; | (SEQ ID NO:43) |
| Phe His Trp Trp Trp Gln Pro; | (SEQ ID NO:44) |
| Gln Pro Ser His Leu Arg Trp; | (SEQ ID NO:45) |
| Ser Pro Ala Ser Pro Val Tyr; | (SEQ ID NO:46) |
| Phe His Trp Trp Trp Gln Pro; | (SEQ ID NO:47) |
| His Pro Ser Asn Gln Ala Ser; | (SEQ ID NO:48) |
| Asn Ser Ala Pro Arg Pro Val; | (SEQ ID NO:49) |
| Gln Leu Trp Ser Ile Tyr Pro; | (SEQ ID NO:50) |
| Ser Trp Pro Phe Phe Asp Leu; | (SEQ ID NO:51) |
| Asp Thr Thr Leu Pro Leu His; | (SEQ ID NO:52) |
| Trp His Trp Gln Met Leu Trp; | (SEQ ID NO:53) |
| Asp Ser Phe Arg Thr Pro Val; | (SEQ ID NO:54) |
| Thr Ser Pro Leu Ser Leu Leu; | (SEQ ID NO:55) |
| Ala Tyr Asn Tyr Val Ser Asp; | (SEQ ID NO:56) |
| Arg Pro Leu His Asp Pro Met; | (SEQ ID NO:57) |
| Trp Pro Ser Thr Thr Leu Phe; | (SEQ ID NO:58) |
| Ala Thr Leu Glu Pro Val Arg; | (SEQ ID NO:59) |
| Ser Met Thr Val Leu Arg Pro; | (SEQ ID NO:60) |
| Gln Ile Gly Ala Pro Ser Trp; | (SEQ ID NO:61) |
| Ala Pro Asp Leu Tyr Val Pro; | (SEQ ID NO:62) |
| Arg Met Pro Pro Leu Leu Pro; | (SEQ ID NO:63) |
| Ala Lys Ala Thr Pro Glu His; | (SEQ ID NO:64) |
| Thr Pro Pro Leu Arg Ile Asn; | (SEQ ID NO:65) |
| Leu Pro Ile His Ala Pro His; | (SEQ ID NO:66) |
| Asp Leu Asn Ala Tyr Thr His; | (SEQ ID NO:67) |
| Val Thr Leu Pro Asn Phe His; | (SEQ ID NO:68) |
| Asn Ser Arg Leu Pro Thr Leu; | (SEQ ID NO:69) |
| Tyr Pro His Pro Ser Arg Ser; | (SEQ ID NO:70) |
| Gly Thr Ala His Phe Met Tyr; | (SEQ ID NO:71) |
| Tyr Ser Leu Leu Pro Thr Arg; | (SEQ ID NO:72) |
| Leu Pro Arg Arg Thr Leu Leu; | (SEQ ID NO:73) |
| Thr Ser Thr Leu Leu Trp Lys; | (SEQ ID NO:74) |
| Thr Ser Asp Met Lys Pro His; | (SEQ ID NO:75) |
| Thr Ser Ser Tyr Leu Ala Leu; | (SEQ ID NO:76) |
| Asn Leu Tyr Gly Pro His Asp; | (SEQ ID NO:77) |
| Leu Glu Thr Tyr Thr Ala Ser; | (SEQ ID NO:78) |
| Ala Tyr Lys Ser Leu Thr Gln; | (SEQ ID NO:79) |
| Ser Thr Ser Val Tyr Ser Ser; | (SEQ ID NO:80) |
| Glu Gly Pro Leu Arg Ser Pro; | (SEQ ID NO:81) |
| Thr Thr Tyr His Ala Leu Gly; | (SEQ ID NO:82) |
| Val Ser Ile Gly His Pro Ser; | (SEQ ID NO:83) |
| Thr His Ser His Arg Pro Ser; | (SEQ ID NO:84) |
| Ile Thr Asn Pro Leu Thr Thr; | (SEQ ID NO:85) |
| Ser Ile Gln Ala His His Ser; | (SEQ ID NO:86) |
| Leu Asn Trp Pro Arg Val Leu; | (SEQ ID NO:87) |
| Tyr Tyr Tyr Ala Pro Pro Pro; | (SEQ ID NO:88) |
| Ser Leu Trp Thr Arg Leu Pro; | (SEQ ID NO:89) |
| Asn Val Tyr His Ser Ser Leu; | (SEQ ID NO:90) |
| Asn Ser Pro His Pro Pro Thr; | (SEQ ID NO:91) |
| Val Pro Ala Lys Pro Arg His; | (SEQ ID NO:92) |
| His Asn Leu His Pro Asn Arg; | (SEQ ID NO:93) |
| Tyr Thr Thr His Arg Trp Leu; | (SEQ ID NO:94) |
| Ala Val Thr Ala Ala Ile Val; | (SEQ ID NO:95) |
| Thr Leu Met His Asp Arg Val; | (SEQ ID NO:96) |
| Thr Pro Leu Lys Val Pro Tyr; | (SEQ ID NO:97) |
| Phe Thr Asn Gln Gln Tyr His; | (SEQ ID NO:98) |
| Ser His Val Pro Ser Met Ala; | (SEQ ID NO:99) |
| His Thr Thr Val Tyr Gly Ala; | (SEQ ID NO:100) |
| Thr Glu Thr Pro Tyr Pro Thr; | (SEQ ID NO:101) |
| Leu Thr Thr Pro Phe Ser Ser; | (SEQ ID NO:102) |
| Gly Val Pro Leu Thr Met Asp; | (SEQ ID NO:103) |
| Lys Leu Pro Thr Val Leu Arg; | (SEQ ID NO:104) |
| Cys Arg Phe His Gly Asn Arg; | (SEQ ID NO:105) |
| Tyr Thr Arg Asp Phe Glu Ala; | (SEQ ID NO:106) |
| Ser Ser Ala Ala Gly Pro Arg; | (SEQ ID NO:107) |
| Ser Leu Ile Gln Tyr Ser Arg; | (SEQ ID NO:108) |
| Asp Ala Leu Met Trp Pro Xaa; | (SEQ ID NO:109) |
| Ser Ser Xaa Ser Leu Tyr Ile; | (SEQ ID NO:110) |
| Phe Asn Thr Ser Thr Arg Thr; | (SEQ ID NO:111) |
| Thr Val Gln His Val Ala Phe; | (SEQ ID NO:112) |
| Asp Tyr Ser Phe Pro Pro Leu; | (SEQ ID NO:113) |
| Val Gly Ser Met Glu Ser Leu; | (SEQ ID NO:114) |
| Phe Xaa Pro Met Ile Xaa Ser; | (SEQ ID NO:115) |
| Ala Pro Pro Arg Val Thr Met; | (SEQ ID NO:116) |
| Ile Ala Thr Lys Thr Pro Lys; | (SEQ ID NO:117) |
| Lys Pro Pro Leu Phe Gln Ile; | (SEQ ID NO:118) |
| Tyr His Thr Ala His Asn Met; | (SEQ ID NO:119) |
| Ser Tyr Ile Gln Ala Thr His; | (SEQ ID NO:120) |
| Ser Ser Phe Ala Thr Phe Leu; | (SEQ ID NO:121) |
| Thr Thr Pro Pro Asn Phe Ala; | (SEQ ID NO:122) |
| Ile Ser Leu Asp Pro Arg Met; | (SEQ ID NO:123) |
| Ser Leu Pro Leu Phe Gly Ala; | (SEQ ID NO:124) |
| Asn Leu Leu Lys Thr Thr Leu; | (SEQ ID NO:125) |
| Asp Gln Asn Leu Pro Arg Arg; | (SEQ ID NO:126) |
| Ser His Phe Glu Gln Leu Leu; | (SEQ ID NO:127) |
| Thr Pro Gln Leu His His Gly; | (SEQ ID NO:128) |
| Ala Pro Leu Asp Arg Ile Thr; | (SEQ ID NO:129) |
| Phe Ala Pro Leu Ile Ala His; | (SEQ ID NO:130) |
| Ser Trp Ile Gln Thr Phe Met; | (SEQ ID NO:131) |
| Asn Thr Trp Pro His Met Tyr; | (SEQ ID NO:132) |
| Glu Pro Leu Pro Thr Thr Leu; | (SEQ ID NO:133) |
| His Gly Pro His Leu Phe Asn; | (SEQ ID NO:134) |
| Tyr Leu Asn Ser Thr Leu Ala; | (SEQ ID NO:135) |
| His Leu His Ser Pro Ser Gly; | (SEQ ID NO:136) |
| Thr Leu Pro His Arg Leu Asn; | (SEQ ID NO:137) |
| Ser Ser Pro Arg Glu Val His; | (SEQ ID NO:138) |
| Asn Gln Val Asp Thr Ala Arg; | (SEQ ID NO:139) |
| Tyr Pro Thr Pro Leu Leu Thr; | (SEQ ID NO:140) |
| His Pro Ala Ala Phe Pro Trp; | (SEQ ID NO:141) |
| Leu Leu Pro His Ser Ser Ala; | (SEQ ID NO:142) |
| Leu Glu Thr Tyr Thr Ala Ser; | (SEQ ID NO:143) |
| Lys Tyr Val Pro Leu Pro Pro; | (SEQ ID NO:144) |
| Ala Pro Leu Ala Leu His Ala; | (SEQ ID NO:145) |
| Tyr Glu Ser Leu Leu Thr Lys; | (SEQ ID NO:146) |
| Ser His Ala Ala Ser Gly Thr; | (SEQ ID NO:147) |
| Gly Leu Ala Thr Val Lys Ser; | (SEQ ID NO:148) |
| Gly Ala Thr Ser Phe Gly Leu; | (SEQ ID NO:149) |
| Lys Pro Pro Gly Pro Val Ser; | (SEQ ID NO:150) |
| Thr Leu Tyr Val Ser Gly Asn; | (SEQ ID NO:151) |
| His Ala Pro Phe Lys Ser Gln; | (SEQ ID NO:152) |
| Val Ala Phe Thr Arg Leu Pro; | (SEQ ID NO:153) |
| Leu Pro Thr Arg Thr Pro Ala; | (SEQ ID NO:154) |
| Ala Ser Phe Asp Leu Leu Ile; | (SEQ ID NO:155) |
| Arg Met Asn Thr Glu Pro Pro; | (SEQ ID NO:156) |
| Lys Met Thr Pro Leu Thr Thr; | (SEQ ID NO:157) |
| Ala Asn Ala Thr Pro Leu Leu; | (SEQ ID NO:158) |
| Thr Ile Trp Pro Pro Pro Val; | (SEQ ID NO:159) |
| Gln Thr Lys Val Met Thr Thr; | (SEQ ID NO:160) |
| Asn His Ala Val Phe Ala Ser; | (SEQ ID NO:161) |
| Leu His Ala Ala Xaa Thr Ser; | (SEQ ID NO:162) |
| Thr Trp Gln Pro Tyr Phe His; | (SEQ ID NO:163) |
| Ala Pro Leu Ala Leu His Ala; | (SEQ ID NO:164) |
| Thr Ala His Asp Leu Thr Val; | (SEQ ID NO:165) |
| Asn Met Thr Asn Met Leu Thr; | (SEQ ID NO:166) |
| Gly Ser Gly Leu Ser Gln Asp; | (SEQ ID NO:167) |
| Thr Pro Ile Lys Thr Ile Tyr; | (SEQ ID NO:168) |
| Ser His Leu Tyr Arg Ser Ser; | (SEQ ID NO:169) |

and His Gly Gln Ala Trp Gln Phe (SEQ ID NO: 170), where Xaa is any amino acid, and is preferably a hydrophobic amino acid.

For covalently linking the javelin to a multi-component viral particle, it may be desirable to add, to the javelin, a “linker region” containing chemical structures which facilitate the linkage reaction. For example, where the javelin is a peptide, a linker region, preferably, but not by way of limitation, containing 1-4 amino acids may be added. As one specific, non-limiting example, where the linking reaction utilizes sulfhydrl groups, a single Cys residue, or a linker peptide such as Cys Gly Ser Gly (SEQ ID NO: 179) may be added to the amino- or carboxy- terminus of a javelin peptide. As another example, a linker may comprise a region that will cause a “kink” in the molecule, such that the javelin may protrude from the surface of the multi-component viral particle; a non-limiting example of such a linker is Pro Gln Pro Gln Pro Lys Pro Gln Pro Gln Pro Gln Pro Lys Pro Gln Pro Lys Pro Glu Pro Glu (SEQ ID NO: 180).

In specific non-limiting embodiments of the invention, where the heat shock protein to be bound is hsp70, BiP and/or members of the hsp70 family the javelin is preferably one of the following peptides, which comprise a javelin region (underlined) and a linker region: Cys Gly Ser Gly His Trp Asp Phe Ala Trp Pro Trp (SEQ ID NO: 171); His Trp Asp Phe Ala Trp Pro Trp Gly Ser Gly Cys (SEQ ID NO: 172); Cys Gly Ser Gly Phe Trp Gly Leu Trp Pro Trp Glu (SEQ ID NO: 173); Phe Trp Gly Leu Trp Pro Trp Glu Gly Ser Gly Cys (SEQ ID NO: 174); Cys His Trp Asp Phe Ala Trp Pro Trp (SEQ ID NO: 175); His Trp Asp Phe Ala Trp Pro Trp Cys (SEQ ID NO: 176); Cys Phe Trp Gly Leu Trp Pro Trp Glu (SEQ ID NO: 177); or Phe Trp Gly Leu Trp Pro Trp Glu Cys (SEQ ID NO: 178).

### 5.3 METHODS OF LINKING VIRAL COMPONENTS TO JAVELINS

A javelin molecule as set forth in the preceding section may be covalently linked to a multi-component viral particle using any method known in the art. Preferably, a plurality ofjavelin molecules are conjugated to each multi-component viral particle. In particular non-limiting embodiments of the invention, a plurality of various species ofjavelin (i.e., javelins having different structures, e.g. different amino acid structures) may be linked to a multi-component viral particle. A multi-component viral particle covalently linked to one or more javelin molecule is said to be “javelinized”.

In determining the method of linking to be used, particular chemical characteristics of the multi-component viral particle may favor the choice of one method over another. For example, where the particle comprises amino-terminal groups, a coupling method which is amino-reactive may be used, and where the particle comprises carbohydrate groups at its surface (e.g., in the context of a glycoprotein), a carbohydrate-based coupling method may advantageously be used (see below).

A pamphlet published by Pierce Chemical Company, entitled “Double Agents™ Cross-Liniking Reagents Selection Guide” (published in 1999, and available from Pierce Chemical Co. as Catalog #1600310) provides a useful set of criteria for selecting a proper agent including the following. Cross-lining reagents are identified by their acronyms, which would be recognized by the skilled artisan. Cleavable and/or non-cleavable cross-linkers may be used. If lysines and sulfhydryl groups are available for cross-linking, one may consider using a heterobifunctional amine/sulfflydryl reactive agent such as AMAS, BMPS, EMCS, sulfo-EMCS, GMBS, sulfo-GMBS, sulfo-KMUS, MBS, sulfo-MBS, SBAP, SIA, SIAB, sulfo-SIAB, SMCC, LC—SMCC, SMPB, SMPH, sulfo-SMPB, SVSB, BMPA, EMCA, KMUA, SMPT, sulfo-LC—SMPT, SPDP, LC—SPDP, and sulfo-LC—SPDP; where the multi-component viral particle is enclosed by or comprises a substantial amount of lipid, it may be desirable, among the foregoing, to utilize membrane-permeable agents such as EMCS, GMBS, MBS, SIAB, SMCC, LC—SMCC, SMPH, SMPT, SPDP, and LC—SPDP. If it is desirable to first react the agent with an —SH group on one molecule (e.g., the javelin) before coupling to an NH₂ on a second molecule (e.g., comprised in the multi-component viral particle), it may be desirable, from among the aforelisted agents, to use BMPA, EMCA or KMUA. If it is desirable to incorporate a carboxyl (COOH) group into one molecule (e.g., the javelin) to facilitate coupling to the second molecule (e.g., comprised in the multi-component viral particle), useful cross-linking reagents may include heterobifunctional, sequential sulfhydryl to amine-reactive agents such as BMPA, EMCA, or KMUA. If one of the components to be linked (e.g., the multi-component viral particle) lacks reactive groups or if the presence or identity of such groups is unknown, it may be desirable to use a heteroflinctional and/or photoreactive cross-linking agent such as ANB—NOS, NHS-ASA, sulfo-NHS-LC-ASA, sulfo-HSAB, SASD, sulfo-SAPB, SANPAH, sulfo-SANPAH, SFAD, ABH, EMCH, KMUH, M₂C₂H, MPBH, ASBA, sulfo-NHS-LC-ASA, SASD, and APDP. From among the agents listed in the preceding sentence, those agents which may be suitable for use in enveloped or high-lipid content multi-component viral particles include ANB—NOS, NHS-ASA, SANPAH, ABH, ASBA, and APDP. Additional information may be found in the Pierce pamphlet and/or in Hermanson, 1995, “Bioconjugate Technologies”, Academic Press, Inc., Pierce Product #20002GJ, and Wong, 1991, “Chemistry of Protein Conjugation and Cross-Linking,” CRC Press,Inc., Pierce Product No. 15010GJ.

In one particular non-limiting set of embodiments, the present invention provides for covalently linking a javelin molecule containing a terminal Cys residue to a multi-component viral particle comprising a terminal NH₂ group (or a javelin molecule containing a terminal NH₂ residue to a multi-component viral particle comprising a terminal Cys residue) using standard techniques, for example, using an amine-sulfhydryl cross-linker such as N-(α-maleimidoacetoxy)-succinimide ester (“AMAS”) or N-(κ-maleimidoundecanoyloxy)-sulfosuccinimide ester (“KMUS”) (Pierce Chemical Co.). Such methods would generally involve reductive methylation of the javelin molecule to block N-termini, cross-linking of blocked peptide at pH 6.5-7.5 using sulfo-KMUS or AMAS, and reacting the succinimide group of the modified javelin with the multi-component viral particle at pH 8-9.

In another non-limiting set of embodiments, the present invention provides for covalently linking ajavelin molecule to a multi-component viral particle via a photo-reactive cross linker. An example of one such cross-linker is N-5-azido-2 nitrobenzyloxy-succinimide (“ANB-NOS”).

In yet another non-limiting set of embodiments, the present invention provides for covalently linking a javelin molecule to a multi-component viral particle via a method which attaches the javelin to a carbohydrate group on the particle. Cross-linking reagents which may be used to effect such linkage include N-(ε-maleimidocaptoyloxy)-succinimide ester (“EMCH”), N-(κ-maleimidoundecanoic acid)hydrazide (“KMUH”), 4-(4-N-maleimidophenyl)-butyric acid hydrazide HCl (“MPBH”), 3-maleimidophenyl boronic acid (“MPBA”) or photoreactive agents (see Pierce Pamphlet, cited supra).

Where one particular method of linkig is appropriate to the multi-component viral particle, the javelin molecules can be engineered to contain a “linker region” containing amino acid residues or other chemical structures which are appropriate to the selected linking method.

Alternatively, if the multi-component viral particle comprises a surface glycoprotein, an oligosaccharide moiety on said glycoprotein may be engineered to express unusual functional groups for selective chemical modification (Mahal et al., 2000, Science 276:1125-1128). The obligate requirement of viruses for the host cell structural and metabolic components, including its post-translational modification machinery, provides a unique opportunity for one skilled in the art to grow and produce viral particles in cell culture in the presence of unnatural derivatives of carbohydrate molecules. For example, depending upon the type of host cell (the term “host” denoting, in part, susceptibility to viral infection), sialic acid is a typical terminal oligosaccharide of most mammalian and viral glycoproteins and glycolipids, with the exception of influenza virus hemagglutinin and neuraminidase. The natural precursor of sialic acid is N-acetylmannosamine. It has been shown by Reutter and co-workers (Kayser et al., 1992, J. Biol. Chem. 267:16934; Keppler et al., 1995, J. Biol. Chem. 27:1308) that, in cell culture and in vivo, unnatural mannosamine derivatives having a substitution of the N-acetyl group are converted into sialosides and incorporated into glycoconjugates. Since functionally reactive ketone groups are virtually absent from naturally occurring amino acids, glycoconjugates and lipids, a ketone group, comprised in a substituent for the N-acetyl group of N-acetylmannosamine, and hence metabolically incorporated into a glycoconjugate comprised in the multi-component viral particle, could provide novel reactive sites for cross-linking.

In yet further embodiments of the invention, a javelin molecule may be incorporated into a multi-component viral particle by the creation of a fusion protein, whereby a nucleic acid encoding a protein component of the particle is engineered to contain, in the proper reading frame, a javelin-encoding nucleic acid. The javelin may be introduced at either terminus or, alternatively, within the body of the protein (e.g., within a protein loop located at the surface of the protein). The nucleic acid may be used to produce its encoded protein using standard techniques.

It may be desirable to vary the number of bound javelin molecules depending on the size of the multi-component viral particle. In particular, it may be desirable to provide a greater number ofjavelin molecules on a larger particle. Where javelins are cross-linked to the particle, this may be achieved by varying the ratio of javelins to particles in the cross-linking reactions. Alternatively, the ratio may be controlled by selecting or engineering the reactive groups for participation in cross-linking; for example, where more javelins are to be bound, a more commonly occurring reactive group may be chosen.

### 5.4 ASSAYS TO DETERMINE LACK OF PATHOGENICITY

It is desirable to confirm that a multi-component viral particle is, in fact, non-pathogenic prior to use. Such determination may be performed before or after the multi-component viral particle is cross-linked to one or more javelin molecules, where cross-linking is the basis for linkage (for example, as opposed to a fusion between ajavelin peptide and a viral protein). Preferably, the determination is performed after the particle is cross-linked to javelin(s), because it is possible that the cross-linking process would alter the degree of pathogenicity or lack thereof.

Because of the obligate intracellular parasitic nature of viruses, a determination of whether or not a multi-component viral particle is pathogenic would generally be performed using a live host cell, tissue, or organism. Non-limiting examples of host systems for evaluating pathogenicity include, but are not limited to, mice, rats and guinea pigs; embryonated chicken eggs, differentiated tissue cultures and cell cultures. The suitability of a host depends on the particle to be tested. For example, where a multi-component viral particle is to be formulated in a vaccine to protect a human subject from infection by a virus X, it may be desirable to test the particle for pathogenicity in a system comprising human cells, such as a cell culture, a tissue culture, a chimeric animal, or a human subject. In this specific example, if the typical host range of infection for virus X includes mice, it may be desirable to first test the particle for pathogenicity in a system comprising murine cells, such as a cell culture, a tissue culture, or a live mouse. If the particle is non-pathogenic to a murine cell, it may then be tested for pathogenicity in a system comprising human cells.

For example, but not by way of limitation, one or more of the following assays can be used, singly or in combination, to evaluate whether a multi-component viral particle is non-pathogenic. As discussed above, to be considered non-pathogenic, a particle should be incapable of replication or replicate (preferably at a slower rate) with little or no pathogenic effects.

As a first specific non-limiting example, pathogenicity of a multi-component viral particle may be tested in mice as follows. Mice (preferably less than 3 days old, an age when they are most susceptible to infection) may be inoculated with varying doses (e.g., between 10⁵ and 10⁷ particles) of a formulation of multi-component viral particles, using a protocol analogous to that described in Allan et al., 1990, J. Immunol. 144:3980-3986 or Allan et al., 1993, Microb. Pathog. 14:75-84. Mice that die on the first day after inoculation may be examined to determine whether they died from inoculation trauma as opposed to particle-induced disease, while surviving mice may be frequently examined thereafter for evidence of pathology. Typical signs of disease include failure to nurse, changes in color or appearance, unusual activities such as excitement, stupor, paralysis or changes in posture. Mice may eventually be sacrificed and tissues may be collected and evaluated by histological examinations. Depending on the particle tested and the virus or viruses from which it (they) is (are) derived, and its (their) pathogenic effects, characteristic effects of retained pathogenicity would be apparent to the skilled artisan.

As a second specific non-limiting example, pathogenicity of a multi-component viral particle may be tested in embryonated chicken eggs. This is particularly appropriate where the particle contains a component derived from an influenza virus, because influenza virus is readily cultivated in embryonated chicken eggs (Burnet, 1936, Br. J. Exp. Pathol. 11:282-293). Multi-component viral particles may be inoculated into the allantoic or amniotic cavity of an embryonated egg. Where the species of particle comprises an influenza virus hemagglutinin component, replication may be detected based on the ability of viral hemagglutinin secreted into the fluid of the amniotic or allantoic cavity to agglutinate chicken erythrocytes. Initially, when such particles are present in lower titers, agglutination of guinea pig, but not chicken erythrocytes may be observed; as titer increases, agglutination of chicken erythrocytes would tend to become more apparent. Alternatively, particularly where the particle does not comprise a hemagglutinin component, replication of particles may be detected by counting the number of lesions produced on the chorioallantoic membrane.

As a third specific non-limiting example, pathogenicity of a multi-component viral particle may be tested in cell culture. Various quantities of a particular formulation of multi-component viral particles may be introduced into a series of cell cultures. Subsequently, pathogenicity may be detected by monitoring the cultures for characteristic changes, known as cytopathic effets (“CPE”), which can typically be readily recognized as foci within a “lawn” of cells (Cooper,1967, Methods in Virology, vol. 3, Academic Press Inc., New York, pp. 243-311). Alternatively, cytopathology may be detected by microscopic examination of the cells for such features as necrosis, formation of intranuclear or cytoplasmic inclusions or the formation of multinucleated giant cells (syncytia). For particles comprised of components derived from viruses that do not cause CPE, cultures may be monitored for other characteristic effects associated with pathogenicity, depending on the parental virus(es). For example, cells infected with particles derived from a noncytopathic, hemagglutinating virus may induce the production of hemagglutinins in the cells or the release of hemagglutinins into the medium (Shelokov,1958, Proc. Soc. Expt. Biol. Med. 97:802-809). In such an example, the adhesion of erythrocytes to the cell surface or the ability of the cell medium to agglutinate erythrocytes may be indicators of pathogenicity.

### 5.5 ASSAYS TO DETERMINE IMMUNOGENICITY

The efficacy of a formulation ofjavelinized multi-component viral particles to induce immunity may be evaluated using assays known in the art to qualitatively or quantitatively assess cellular immune responses and/or humoral (antibody-mediated) responses. Traditional vaccines and adjuvants stimulate antibody production, which can include the generation of cytotoxic antibodies. However, a biologically more efficient method for the elimination of virally infected cells is the cellular immune response, which utilizes cytotoxic T lymphocytes, T-cells which have evolved to recognize and kill diseased cells. Therefore, it is preferable that a formulation of javelinized multi-component viral particles induces cellular immunity. The level of immunity required to protect against or diminish the symptomatology of infection varies from virus to virus, but is generally known in the art.

The ability to induce humoral immunity may be determined using standard techniques. For example, serum produced by a human inoculated with a formulation ofjavelinized multi-component viral particles may be tested in an ELISA assay (Engvall and Perlman,1971, Immunochemistry 8:871-879; Van Vunakis and Langone, eds., 1980, Methods Enzymol. 70:1-525) using a surface protein of the virus towards which immunity is desired (herein, the “target virus”) as target antigen and an enzymatically labeled anti-human immunoglobulin antibody as a detection reagent. Alternatively, the ability of antiserum to neutralize viral infectivity may be tested.

The following is a specific, non-limiting example of how an ELISA assay may be used to assay the humoral immune response developed in mice toward a formulation of javelinized multi-component viral particles. Mice may be immunized with a formulation ofjavelinized multi-component viral particles (e.g., between about 1×10⁵ and 1×10⁶ particles) by a suitable route (such animals are “test-immunized”). Choice of a suitable route may be made by considering the typical route of infection of the target virus toward which the javelinized particle fonnulation is intended to induce immunity. For example, where the target virus is a respiratory virus, intra-nasal, or intra-tracheal, immunization may be preferred. Where the target virus typically infects by the oral-fecal route, oral immunization may be desirable. It should be noted, however, that routes not typically used by the target virus may also be used, so that, depending upon the circumstances, any standard route of immunization may be appropriate. For positive controls, mice may be infected with the unjavelinized parent “target virus” (e.g., between about 1×10⁵ and 1×10⁶ pfu) by a route similar to that used for immunization (such animals are “control-infected”). Uninfected, non-immunized mice may be used as negative controls. The presence of serum antibodies produced against known viral antigens or viral lysate may be monitored 7 days post-immunization using an ELISA. For example, microtiter plates may be coated overnight at 4° C. or for 2 h at 37° C. with approximately 10 μg/mL of purified or partially purified antigen. Unbound antigen may be removed by extensive washing. Serum from the mice at various dilutions may then be added and incubated for at least 1 h at 37° C., and then may be washed extensively to remove unbound antibodies present in the serum. A second antibody, reactive with antibodies from the serum (here, anti-mouse immunoglobin antibody) coupled with, for example, horseradish peroxidase, alkaline phosphatase or urease may then be added and incubated for at least another 1 h at 37° C. The microtiter plates may be washed once again before addition of enzyme specific substrate. Absorbance to detect modified substrate may be measured using a microtiter plate spectrophotometer. The serum antibody titer may then be determined from a standard curve.

The ability of a formulation of javelinized multi-component viral particles to induce cellular immunity may be determined by a cytotoxic T lymphocyte assay. As a specific, non-limiting example of such an assay, the induction of cellular immunity in mice may be tested by inoculating, by a suitable route (see supra) mice withjavelinized multi-component viral particles (e.g., between about 1×10⁵ and 1×10⁶ particles) which comprise a component from a target virus (to produce “test-immunized” mice, see supra). For positive controls, mice may be challenged with parental target virus (e.g., between about 1×10⁵ and 1×10⁶ pfu) by a similar inoculation method (to produce “control-infected” mice, see supra). Uninfected, non-immunized mice may be used as negative controls. Effector T cells or mixed lymphocytes may then be collected from test- immunized, control-infected and negative control mice by bronchoalveolar lavage (BAL) or from spleens or lymph nodes from animals sacrificed 10 days after the last inoculation. Effector CTLs, especially CD8⁺ T cells, may be further enriched by culturing the collected cells in vivo for 5 days in RPMI medium, 10% FCS, penicillin-streptomycin and 2 mM L-glutamine together with 4×10⁴ γ-irradiated (3,000 rads) stimulator cells infected with the parental target virus, transfected with the appropriate gene or treated with a virus-specific peptide or mitomycin C. The presence of CTL activity may then be detected by measuring the ability of the collected cells to lyse target cells which are perceived by the effector cells as being virally infected, either as a result of bonafide infection or coating with viral peptides. Target cells may be prepared by either infecting susceptible cells (e.g., at a multiplicity of infection of 10) or by coating cells with viral peptides that are known to induce a CTL response (e.g., at a concentration of 0.1-100 μg/ml). The target cells may then be labeled for 1 h with 250 mCi/ml of ⁵¹Cr (sodium chromate) in Tris-Phosphate buffer, pH 7.4 at 37° C. for 60 minutes. After washing to remove free chromium, ⁵¹Cr-labeled target cells may be mixed with effector lymphocytes to yield several different Effector:Target (E:T) ratios (e.g., between about 1:1 and 100:1), after which the cultures may be incubated for 6 h. Supernatants may then be harvested and the radioactivity in the medium due to cell lysis resulting from an effector T cell immune response to target cells may be measured in a gamma counter. Percent specific lysis is calculated as 100×[(cpm release by CTL−cpm spontaneous release)/(cpm maximal release−cpm spontaneous release)]. Maximal release may be determined by addition of 1% Triton X-100 to the target cells. The effectiveness ofjavelinized multi-component viral particles to produce a cytotoxic T cell response may be compared with that of target virus by comparing the percent specific lysis of effector cells harvested from test-immunized mice with that produced by effector cells harvested from control-infected mice and from negative control mice. In preferred, non-limiting embodiments of the invention, the magnitude of the cytotoxic T cell response induced by the javelinized multi-component viral particle formulation is at least between 2-5 times greater than the negative control value.

Another, more recently developed assay for the enumeration of antigen specific CTLs is the tetramer assay as described in McHeyzer-Williams, 1995, Science 268:106-111; Altman et al.,1996, Science 274:94-96; Murali-Krishna et al., 1998, Immunity 8:177-187; Flynn et al., 1998, Immunity 8:683-691; International Patent Application No. PCT/US97/04694, Publication No. WO 97/35991, by The Johns Hopkins University, Schneck et al., inventors; U.S. Pat. No. 5,635,363 by Altman et al., issued Jun. 3, 1997; International Patent Application No.

PCT/US96/02606, Publication No. WO 96/26962 by The Board Of Trustees of Leland Stanford Junior Univeristy, Altman et al., inventors; and U.S. Pat. No. 6,015,884 by Schneck and O'Hernin, issued Jan. 18, 2000. Tetramers are MHC Class I molecules that have been generated in vitro to contain a nominal antigenic peptide and can bind to T cell receptors that have been primed. As a specific, non-limiting example, a CTL response in mice developed toward a formulation ofjavelinized multi-component viral particles may be assayed as follows. Mice may be inoculated, by a suitable route, with a formulation ofjavelinized multi-component viral particles (e.g., between about 1×10⁵ and 2×10⁶ particles). Specific cytotoxic T lymphocytes may then be enumerated from spleen cells freshly explanted from test-immunized, control-infected, and negative control mice by the binding of tetrameric complexes to the cells 8 days post-infection. Analysis of binding is carried out by flow cytometry. The magnitude of the cytotoxic T cell response is believed to be directly proportional to the amount of tetramer binding.

Another method for evaluating the immunogenicity of formulations of multi-component viral particles involves intracellular staining for cytokines such as interferon γ and TNF α. Both cell mediated and humoral responses to the javelinized viral vaccines can be evaluated by the detection of cytokine production either intracellularly or in secreted form. One specific, non-limiting example of how such a technique may be used to determine the immune response produced in mice is as follows. Mice may be inoculated with a formulation of javelinized multi-component viral particles (e.g., between about 1×10⁵ and 1×10⁷ particles) by a suitable route (to produce “test-immunized” mice). For positive controls, mice may be infected with target virus (e.g., 1-10×10⁶ pfu) using a similar method of immunization as was used for the javelinized particle formulation (to produce “control-infected” mice). Uninfected, non-immunized “naive” mice may be used as negative controls. Eight days post-inoculation or infection spleen cells may then be prepared from test-immunized, control-infected and negative control mice and cultured for 5 h in 96-well flat-bottomed plates at a concentration of 1×10⁶ cells/well in 0.2 mL complete medium supplemented with 10 units/well recombinant human IL-2 and 1 μL/mL Brefeldin A with or without target virus and/or target virus antigens. Following 5 h of culture, cells may be harvested, washed once with PBS buffer and surface-stained with phycoerythrin (PE)-conjugated monoclonal rat anti-mouse CD8a (clone 53-6.7) antibody and APC-conjugated CD4 antibody. Both antibodies may be obtained from Pharmingen, San Diego, Calif. After 30 minutes of incubation at 4° C., unbound antibody may be removed from the cells by washing with PBS, and the cells may be subjected to intracellular cytokine staining using Cytofix/Cytoperm according to the manufacturer's instructions (Pharmingen, San Diego, Calif.). For intracellular interferon (IFN) γ staining, fluorescein (FITC)-conjugated monoclonal antibody rat anti-mouse IFN γ antibody (clone XMG 1.2) and its isotype control antibody (rat IgG1) (both antibodies are from Phanningen, San Diego, Calif.) may be used. Intracellular TNF α may be stained using FITC-conjugated anti-TNF α antibody. Three to four color flow analyses (Tripp et al., 1995, J. Immunol. 154:5870-5875) of cells stained with various combinations of fluorochrome-conjugated monoclonal antibodies for CD4, CD8, other cell surface markers and various cytokines such as IFN γ and TNF α may be carried out using a fluorescent-activated flow cytometer such as FACsCalibur, available from BD Biosciences, San Jose, Calif.). In preferred, non-limiting embodiments of the invention, the magnitude of the cytokine response induced by the javelinized multi-component viral particle formulation is at least between 2 and 5 times greater than the negative control.

As yet another methodology, cytologic changes in tissues associated with viral infections can be used diagnostically to determine the efficacy of a formulation of multi-component viral particles in inducing an immune response. These cytologic changes can be determined using various staining methods or visualized using specific antibody against viral antigens and immunofluorescent microscope or electron microscopy. As a specific, non-limiting example, the induction of an immune response in mice by a formulation of multi-component viral particles may be tested as follows. Mice may be inoculated with a formulation of multi-component viral particles (e.g., about 1-2×10⁵ particles) by a suitable route and may then be challenged with target virus (e.g.,1-10×10⁶ pfu ) to produce “test-infected-immunized” mice,. Non-immunized mice that are infected with virus may be used as positive controls (as exhibiting a cytopathic effect), and uninfected, non-immunized naive mice may be used as negative controls. Test-infected-immunized, positive control and negative control animals may then be sacrificed and the various tissues examined for any changes at the cellular level. Alternatively, the animals may be observed for the manifestation of symptoms of disease or the absence thereof. Analogously, cell cultures may be used and analyzed for plaque formation.

Methods analogous to those set forth above in this section may be used to evaluate the immune response produced by any species, including humans.

### 5.6 COMPOSITIONS OF THE INVENTION

The present invention provides for formulations comprising javelinized multi-component viral particles in amounts effective in inducing a protective immune response toward a target virus. A “protective” immune response is one which either prevents infection, substantially reduces the duration or severity of infection, and/or increases baseline immunity to the target virus (humoral and/or cellular) by a factor of at least between about 2 and 10 fold.

The formulations of the invention may comprise one species of javelinized multi-component viral particles or a plurality of species of javelinized multi-component viral particles. A single species ofjavelinized multi-component viral particle is defined herein as a group of particles having the same sub-components, but where the proportions and linkage of sub-components may vary between particles. For example, a single species ofjavelinized multi-component viral particles may comprise an attenuated strain of HSV, cross-linked, via amino terminal groups, to three different types ofjavelin molecules, termed types “C”, “D”, and “E”. While the exact proportions and locations ofjavelins C, D and E may vary between particles, the particles all belong to the same species.

Where a formulation comprises a plurality of species of javelinized multi-component viral particles, the species may differ in the types ofjavelins linked to a single type of multi-component viral particle (e.g., where a multi-component viral particle comprising cross-linked gp120, env and protease proteins of HIV-1 is further cross-linked to javelins C, D and E to form one species, or alternatively the same type of particle is further cross-linked to javelins F, G and H to form another species); in the strain of virus represented (e.g., one species may comprise an attenuated strain of HPV-18 conjugated to javelin I, and another species may comprise an attenuated strain of HPV-16 conjugated to javelin I); in the type of virus represented (where viruses which are sufficiently structurally different as to not be considered different strains or mutants or variants are different “types”; e.g., one species may comprise cross-linked HIV-1 gp160 and env proteins further cross-linked to javelins J and K, and another species may comprise killed HSV 8 cross-linked to javelins J and K), or may vary in any combination of the foregoing characteristics or other structural differences (e.g. one species is an attenuated strain of influenza cross-linked to javelins L and M, another species is a killed RSV virus cross-linked to javelin N, and another species is Hepatitis B surface antigen cross-linked to a fusion protein between tetanus toxoid and javelin O).

Formulations of the invention may, in certain embodiments, further comprise one or more type of heat shock protein to which the javelinized multi-component viral particles are intended to bind. Such heat shock proteins may be prepared from natural sources, be synthesized chemically, or be produced using standard biotechnology methodologies. A single species or a plurality of species of heat shock proteins may be incorporated into formulations according to the invention.

For example, cDNAs which may be used to express heat shock proteins include, but are not limited to, gp96: human: Genebank Accession No. X15187; Maki et al., Proc. Natl. Acad. Sci. U.S.A. 87:5658-5562; mouse: Genebank Accession No. M1 6370; Srivastava et al., Proc. Natl. Acad. Sci. U.S.A. 84:3807-3811; BiP: human: Genebank Accession No. M19645, Ting et al., 1988, DNA 1:275-286; mouse Genebank Accession No. U16277, Haas et al., 1988, Proc. Natl. Acad. Sci. U.S.A. 85:2250-2254; hsp70: human: Genebank Accession No. M24743, Hunt et al., 1985, Proc. Natl. Acad. Sci. U.S.A. 82:6455-6489; mouse: Genebank Accession No. M35021, Hunt et al., 1990, Gene 87:199-204; and hsp40: human: Genebank Accession No. D49547, Ohtsuka, 1993, Biochem. Biophys. Res. Commun. 197:235-240. Such sequences may be expressed using any appropriate expression vector known in the art. Suitable vectors include, but are not limited to, herpes simplex viral based vectors such as pHSV1 (Geller et al., 1990, Proc. Natl. Acad. Sci. U.S.A. 87:8950-8954); retroviral vectors such as MFG (Jaffee et al., 1993, Cancer Res. 53:2221-2226), and in particular Moloney retroviral vectors such as LN, LNSX, LNCX, and LXSN (Miller and Rosman, 1989, Biotechniques 2:980-989); vaccinia viral vectors such as MVA (Sutter and Moss, 1992, Proc. Natl. Acad. Sci. U.S.A. 89:10847-10851); adenovirus vectors such as pJM17 (Ali et al., 1994, Gene Therapy 1:367-384; Berker, 1988, Biotechniques 6:616-624; Wand and Finer, 1996, Nature Medicine 2:714-716); adeno-associated virus vectors such as AAV/neo (Mura-Cacho et al., 1992, J. Immunother. 11:231-237); pCDNA3 (InVitrogen); pET 11a, pET3a, pET11d, pET3d, pET22d, and pET12a (Novagen); plasmid AH5 (which contains the SV40 origin and the adenovirus major late promoter); pRC/CMV (InVitrogen); pCMU II (Paabo et al., 1986, EMBO J. 5:1921-1927); pZipNeo SV (Cepko et al., 1984, Cell 37:1053-1062) and pSRα (DNAX, Palo Alto, Calif.).

If a heat shock protein is comprised in a formulation according to the invention, it may be desirable to “load” such heat shock proteins with javelinized multi-component viral particles prior to administration to a subject. For example, such formulations may be prepared by incubating heat shock protein with javelinized multi-component viral particles in the presence of ADP. For example, but not by way of limitation, in such incubations the concentration of ADP may be between about 0.5 and 5 mM. The preformed complex may be stored at −80° C., −4° C., preferably at −20° C., and more preferably at 4° C. In preferred, non-limiting embodiments of the invention, the complex may be formed 30 to 60 minutes prior to administration. Alternatively, the complex or individual components thereof may be stored in lyophilized form and reconstituted prior to use.

The formulations of the present invention may further comprise conventional adjuvants and/or cytokines, such as GM-CSF.

The formulations of the invention may comprise a pharmaceutically suitable carrier, such as, but not limited to, normal saline. The formulations of the invention may also utilize delivery systems as are known in the art, for example, liposome and microsphere delivery systems.

The foregoing formulations, for administration to a subject in need of such treatment (see infra), are also referred to herein as “vaccines”. The use of the term “vaccine” is not intended to indicate that complete protection from infection is necessarily afforded, but rather that a “protective immune response”, as defined above, is achieved.

### 5.7 METHODS OF PROVIDING IMMUNITY

The present invention provides for methods of providing a protective immune response in subjects in need of such treatment. Depending upon the target virus, such subjects may constitute a general population (for example, it is recommended that all children be vaccinated against polio) whereas in other instances, only a risk group may be desirably immunized (e.g., a person traveling into an area where a particular virus, such as, for example, tick-borne encephalitis or ebola virus, is endemic). The subject may be a human or a non-human subject.

The route of inoculation may be varied depending on the nature of the target virus (for example, in certain cases it may be desirable and feasible to induce an immune response at the site where the immune system first encounters the virus, e.g., mucosal immunity). Suitable routes of inoculation include, but are not limited to, intra-nasal, intramuscular, denmal, subdermal, subcutaneous, oral, intra-tracheal, intravenous, intraperitoneal, and intrathecal.

The dosage of formulation may vary depending upon the target virus and the javelinized multi-component viral particle formulation to be used. It may also vary based on the route of administration, the bioavailability of the particles, and the size of the subject to be vaccinated. In specific, non-limiting embodiments of the invention, the number ofjavelinized multi-component viral particles may range from 100,000 to 10,000,000, and is preferably 1000-20,000 particles.

Preferably, the formulation of the invention is used to inoculate a subject and then, after a suitable period of time, a second inoculation of the formulation is administered as a “booster”. Additional “boosters” may be administered, as necessary to provide and/or maintain suitable levels of immunity.

Various publications are cited herein, the contents of which are hereby incorporated by reference in their entireties.

What is claimed is:

1. An immunogenic complex comprising a multi-component viral particle covalently linked to ajavelin molecule, wherein the javelin molecule is a peptide which selectively binds to a heat shock protein.

2. The immunogenic complex of claim 1, wherein the multi-component viral particle is an attenuated virus.

3. The immunogenic complex of claim 1, wherein the multi-component viral particle is a killed virus.

4. The immunogenic complex of claim 1, wherein the multi-component viral particle is derived from an influenza virus.

5. The immunogenic complex of claim 1, wherein the multi-component viral particle is derived from a human immunodeficiency virus.

6. The immunogenic complex of claim 1, wherein the multi-component viral particle is derived from a herpes simplex virus.

7. The immunogenic complex of claim 1, wherein the multi-component viral particle is derived from a human papilloma virus.

8. An immunogenic composition, comprising a plurality of complexes each comprising a multi-component viral particle covalently linked to a javelin molecule, wherein the javelin molecule is a peptide which selectively binds to a heat shock protein.

9. The immunogenic composition of claim 8, wherein the multi-component viral particles are derived from the same strain of virus.

10. The immunogenic composition of claim 8, wherein the multi-component viral particles are derived from different strains of virus.

11. The immunogenic composition of claim 8, wherein the multi-component viral particles are derived from the same type of virus.

12. The immunogenic composition of claim 8, wherein the multi-component viral particles are derived from different types of virus.

13. The immunogenic composition of claim 12, wherein the multi-component viral particles are derived from a human immunodeficiency virus and a herpes simple virus.

14. The immunogenic composition of claim 8, comprising a multi-component viral particle which is an attenuated virus.

15. The immunogenic composition of claim 8, comprising a multi-component viral particle which is a killed virus.

16. The immunogenic composition of claim 8, comprising a multi-component viral particle which is derived from an influenza virus.

17. The immunogenic composition of claim 8, comprising a multi-component viral particle which is derived from a human immunodeficiency virus.

18. The immunogenic composition of claim 8, comprising a multi-component viral particle which is derived from a herpes simplex virus.

19. The immunogenic composition of claim 8, comprising a multi-component viral particle which is derived from a human papilloma virus.

20. The immunogenic composition of claim 8, further comprising an effective amount of a heat shock protein,

21. A method of inducing an immune response to a target virus is a subject, comprising administering, to the subject, an effective amount of an immunogenic composition comprising complexes comprising multi-component viral particles covalently linked to a javelin molecule, wherein the javelin molecule is a peptide which selectively binds to a heat shock protein, and wherein the multi-component viral particle is derived from the target virus.
